(19) United States
(12) Patent Application Publication
(10) Pub. No.: US 2003/0109860 A1
(43) Pub. Date: Jun. 12, 2003

(54) Multiple laser treatment

(76) Inventor: Michael Black, Foster City, CA (US)

Correspondence Address: COOLEY GODWARD LLP, ATTN: PATENT GROUP, ONE FREEDOM SQUARE, RESTON TOWN CENTER 11951 FREEDOM DRIVE, RESTON, VA, 20190-5601, US

(21) Appl. No.: 10/017,287

(22) Filed: Dec. 12, 2001

(51) Int. Cl. A61B 18/18

(52) U.S. Cl. 606/10; 606/13; 606/17; 607/89

(57) ABSTRACT

A multiple laser treatment apparatus and method is provided that includes two or more lasers. Each laser simultaneously delivers a laser treatment beam. Each laser treatment beam has at least one distinct laser beam parameter. Different means to select two or more laser treatment beams and laser beam parameters are included. The present invention further includes means to deliver the laser treatment beams in a combined treatment beam. The combined treatment beam is delivered at a substance at which the substance undergoes treatment. The present invention further includes a computer program to control and manage the simultaneous delivery of multiple laser treatment beams to a substance. Furthermore, a database that contains of a plurality of laser treatment plans is provided. The present invention enables one to simultaneously deliver a combined treatment beam to a substance with the greatest variety according to the need of a treatment of the substance.

### CROSS-REFERENCE TO RELATED APPLICATIONS

This application is cross-referenced to copending U.S. patent application entitled “Multiple Laser Diagnostics” by inventor Michael Black with filing date Dec. 12, 2001, which is hereby incorporated by reference.

### FIELD OF THE INVENTION

This invention relates generally to laser systems. More particularly, the present invention relates to multiple laser treatment systems.

### BACKGROUND

Lasers have many useful applications to the treatment of surfaces. For example, laser heat treating of metals has become a valuable industrial process since it provides a way for selectively hardening specific areas of a metal part. Lasers have also become valuable medical instruments. Medical laser systems in the prior art teach different types of lasers that produce light beams with different wavelengths to be used for various types of surgical and medical applications. The success of medical laser systems in these applications is dependent on, for instance, the wavelength of the laser and the interaction of the laser with the tissue.

The absorption of light energy produced by a laser is dependent on the characteristics of the tissue. Since human tissue is approximately 80%-90% water, the absorption of radiation energy (i.e. light energy) in water will determine the characteristics of laser interaction in tissue. For instance, the CO₂ laser has been found to provide a very good “light knife” due to its ability to induce incisions with less charring with good hemostatic control. However, the Nd:YAG laser has better photocoagulative ability, as its 1.064 micron wavelength penetrates much deeper into tissue than the 10.6 micron radiation, and is closer to the hemoglobin absorption peak (i.e. approximately 0.577 microns). Because the water absorption peak has been found to be approximately 2.9 microns, the Er:YAG laser is of special interest as providing an optimum “light knife” whose light beam wavelength is much closer to the absorption peak of hemoglobin (i.e. blood) than the CO₂ laser, and should theoretically provide better coagulative effects in conjunction with its superb cutting abilities. In practice, however, it has been observed that Er:YAG radiation is absorbed so strongly by the water content of the tissue that it provides very poor hemostasis. Currently, various commercial lasers have been used for surgical treatments and include gas lasers (such as CO₂, excimer, argon, cu— vapor lasers), liquid lasers (dye lasers) and solid state lasers (such as YAG, semiconductor, Ti:sapphire lasers).

According to the various types of surgical and medical laser applications, the prior art teaches different ways of utilizing a single laser system to vary or switch to another single wavelength that is useful for a particular application. For instance, U.S. Pat. No. 5,144,630 to JJT International, Inc. discusses that for medical applications which are governed mainly by the laser beam's wavelength, there is a strong need for a multiple-purpose laser system in which multi-wavelengths may be generated from a single laser unit. In that light, U.S. Pat. No. 5,144,630 discloses a process and apparatus for selecting multi-wavelength coherent radiations ranging from deep-ultraviolet to mid-infrared using a single solid state laser by switching to the appropriate frequency converters which are integrated in one laser unit. U.S. Pat. No. 6,096,031 to Coherent, Inc. discloses a medical laser system for ablating biological material using a solid state laser. In addition, they disclose the use of multiple lasers to accelerate the power of the medical laser system for such an application. U.S. Pat. No. 6,162,213 to Cincinnati Sub-Zero Products, Inc. discloses a laser system with a single laser for producing a fifth harmonic generation beam of a predetermined wavelength, eliminating the difficulties of alignment of two separate laser beams. U.S. Pat. No. 6,162,213 teaches that one or more than one multi-wavelength may be selected by switching to the appropriate frequency converters which are integrated in one laser unit. The prior art also teaches different ways of utilizing a laser system with multiple lasers to deliver multiple wavelengths that are useful for a particular application. U.S. Pat. No. 5,139,494 to Premier Laser Systems Inc. teaches a medical system wherein different lasers transmit multiple wavelengths to a tissue site. However, each laser in this medical system transmits a different and predetermined wavelength. Furthermore, U.S. Pat. No. 5,139,494 mentions that the wavelengths are transmitted along a common optical pathway, however, there is no teaching to how this is established. U.S. Pat. Nos. 5,655,547 and 5,970,983 both to ESC Medical Systems Ltd. teach a method of selecting a coherent radiation source for ablating and selecting a coherent radiation source for coagulating for skin tissue and soft dental tissue respectively. U.S. Pat. Nos. 5,655,547 and 5,970,983 both mention that the ablating beam is directed substantially simultaneously with the coagulating beam, however, there is no teaching related to how this is accomplished.

Most laser delivery systems employ refractive lens-based optics to guide and focus the laser beam. However, any laser delivery system based on the use of optical lenses is unequivocally dedicated only to one predetermined laser source wavelength due to chromatic aberration (See for instance, U.S. Pat. No. 4,917,083). This means that each time the user wants to change the laser wavelength, for example, for changing the type of a surgical procedure, the user has to replace the laser beam delivery apparatus.

With the advancement of medical laser treatments, in particular surgical and endoscopic procedures, it is not uncommon that a patient with a particular complaint or disease will have to undergo several laser treatments wherein, for instance, each treatment could require a different laser and specifies, for instance, a particular wavelength, spot size and energy to obtain a desired result for that disease or complaint. The current laser treatment systems that utilize a laser system to vary or switch to another single wavelength are then becoming less effective and increasingly time consuming when the level of sophistication of laser treatment increases. Accordingly, there is a need to develop advanced medical laser treatment systems that are versatile and match the current needs of surgical and endoscopic procedures with the greatest variety.

### SUMMARY OF THE INVENTION

The present invention provides a multiple laser treatment apparatus and method that overcomes the limitations of prior art developments and methods. The present invention provides a versatile and flexible system that meets the current needs of laser treatments with the greatest variety.

A multiple laser treatment apparatus and method of the present invention includes n lasers. Each laser simultaneously delivers a laser treatment beam. Each laser treatment beam has at least one distinct laser beam parameter. Each laser beam parameter is selected for a treatment. In general, the present invention includes two or more lasers. The lasers can be different lasers or the same type of lasers. In case of the same type of laser at least one laser beam parameter in each laser treatment beam is different. In general, one or more laser beam parameters of the laser treatment beams are different. However, one or more laser beam parameters of the laser treatment beams can also be the identical.

Examples of laser beam parameters are provided and include, for instance, wavelengths, fluences, power levels, energy levels, temporal parameters, geometrical parameters, spot sizes, linear delivery parameters or three-dimensional delivery parameters. A spectrum of wavelengths can be selected ranging from ultraviolet to far infrared. As one skilled in the art might readily appreciate, a large number of combinations of laser beam parameters could be derived even if just two of the same lasers are used.

The present invention provides different means to select two or more laser treatment beams and laser beam parameters. For instance, at least one optical component could be included to adjust or control one or more laser beam parameters of one or more of laser treatment beams. Examples of optical component are, for instance, but not limited to, a beam profiler, a collimator, a spherical element, an a-spherical element or a parabolic element. In addition, the means to select also includes means to control each one of the lasers. Each laser can be controlled separately or by an overarching single control panel. The present invention also includes means to control one or more laser beam parameters of at least one of the laser treatment beams.

The present invention further includes means to deliver the laser treatment beams in a combined treatment beam. Subsequently, the combined treatment beam is delivered at a substance at which the substance undergoes treatment. Treatment in the present invention is then defined as a combination of two or more different laser treatment beams applied simultaneously. The type of treatment is dependent on the substance and the structural change of the substance that one wants to achieve. The substance in the present invention is, for instance, but not limited to, a biological tissue, a (bio)chemical compound, a bioengineering composition, a fluid, a food product or a physical structure. An example of a treatment is a medical treatment and the laser treatment beams in the combined treatment beam are medically useful treatment beams.

The means to deliver could include a mirror-based optical delivery device to control the combined treatment beam. The mirror-based optical delivery device could include linear delivery means and/or three-dimensional delivery means. The means to deliver could also include a micromanipulator, endoscopic delivery means or an optical device.

The present invention further includes means for diagnosing a substance. A diagnosing means includes a diagnostic system, wherein the diagnostic system is capable of mapping an area of the substance using fluorescent emission. This map can be used, for instance, to recommend a treatment plan.

The apparatus of the present invention could be a handheld delivery apparatus. The handheld delivery apparatus is then a portable and transferable miniature handheld delivery apparatus with, for instance, dimensions of 6″ by 12″ by 20″ or less. Such a handheld apparatus operates on an independent power source such as battery power.

In general, the method of the present invention for simultaneously delivering a combined laser treatment beam includes the step of selecting two or more laser treatment beams with each laser treatment beam having at least one different laser beam parameter. The method further includes the step of simultaneously delivering the laser treatment beams in a combined laser treatment beam to a substance at which the substance undergoes a treatment.

The present invention also includes a computer program to control and manage the simultaneous delivery of multiple laser treatment beams to a substance. The computer program includes means for selecting a treatment plan. The treatment plan includes two or more laser treatment beams with each laser treatment beam having at least one different laser beam parameter. Different examples are provided for selecting a treatment plan. For instance, a treatment plan could be recommended, a treatment plan could be obtained from a database, or a treatment plan could be compared with a treatment plan that was used in a previous treatment plan. The computer program further includes means for entering data. Different type of data could be entered such as, for instance, patient data, treatment plan data, or complaint or disease data. The computer program also includes means for applying the treatment plan to the substance. However, before a treatment plan is applied to a substance the computer program also includes means to (optionally) verify the treatment plan. The computer program also includes communication means to communicate information between the computer program and one or more remote stations or users.

Furthermore, the present invention includes a database that contains of a plurality of laser treatment plans. Each treatment plan lists two or more laser treatment beams that could be delivered simultaneously to a substance. The treatment plans are, for instance, medical treatment plans, (bio)chemical treatment plans or physical treatment plans. The database is not limited to treatment plan information as it could also include information that is substance-related or patient-related.

In view of that which is stated above, it is the objective of the present invention to provide an apparatus and method that is able to deliver a combined treatment beam to a substance with the greatest variety according to the need of a treatment of a substance.

It is another objective of the present invention to provide a multiple laser treatment apparatus and method to simultaneously deliver two or more laser treatment beams as a combined laser treatment beam.

It is yet another objective of the present invention to provide a multiple laser treatment apparatus and method wherein each laser treatment beam in the combined laser treatment beam has at least one distinct laser beam parameter.

It is still another objective of the present invention to provide a multiple laser treatment apparatus and method wherein each laser treatment beam in the combined laser treatment beam has a distinct wavelength ranging from ultraviolet to far infrared.

It is still another objective of the present invention to provide means to select two or more laser treatment beams and laser beam parameters.

It is still another objective of the present invention to provide optical components to alter or control one or more laser beam parameters of one or more laser treatment beams in the combined laser treatment beam.

It is still another objective of the present invention to provide control of two or more lasers.

It is still another objective of the present invention to provide control of one or more laser beam parameters.

It is still another objective of the present invention to preserve the mode of each laser treatment beam.

It is still another objective of the present invention to provide a mirror-based delivery device to control the combined laser treatment beam.

It is still another objective of the present invention to provide a multiple laser treatment apparatus and method with linear scanning and delivery capability of the combined laser treatment beam.

It is still another objective of the present invention to provide a multiple laser treatment apparatus and method with three-dimensional scanning and delivery capability of the combined laser treatment beam.

It is still another objective of the present invention to provide a computer program to control and manage the simultaneously delivery of multiple laser treatment beams to a substance with a multiple laser treatment apparatus and method.

It is still another objective of the present invention to provide a database of treatment plans for laser applications wherein two or more laser treatment beams are simultaneously delivered to a substance.

It is still another objective of the present invention to diagnose and map an area of a substance using fluorescent emission and to use this map to recommend a treatment plan.

The advantage of the present invention over the prior art is that the apparatus enables one to perform a treatment plan with the greatest variety of laser treatment beams at the same time. Another advantage of the present invention is that it enables one to deliver two or more different laser treatment beams simultaneously in a combined beam to a substance wherein each laser treatment beam has at least one different laser beam parameter. Yet another advantage of the present invention is that it significantly decreases the overall laser treatment and operation time. Still another advantage of the present invention is that it provides for the means to advance laser treatment plans or recipes with combined laser treatment beams for simultaneous delivery to a substance.

### BRIEF DESCRIPTION OF THE FIGURES

The objectives and advantages of the present invention will be understood by reading the following detailed description in conjunction with the drawings, in which:

FIG. 1 shows an example of a multiple laser treatment apparatus and method according to the present invention;

FIG. 2 shows an example of a multiple laser treatment apparatus and method wherein optical components are included to select the laser beam parameters according to the present invention;

FIG. 3 shows an example of a multiple laser treatment apparatus and method with means to control according to the present invention;

FIGS. 4-7 shows different examples of two different laser treatment beams in a combined beam according to the present invention;

FIG. 8 shows an optical device to select and combine laser treatment beams according to the present invention;

FIG. 9 shows a mirror-based delivery means;

FIG. 10 shows a flow diagram of a computer program according to the present invention;

FIG. 11 shows an illustration of a communication system between the apparatus and method of the present invention and remote agents; and

FIG. 12 shows an example of a multiple laser treatment apparatus and method including a diagnosing means according to the present invention.

### DETAILED DESCRIPTION OF THE INVENTION

Although the following detailed description contains many specifics for the purposes of illustration, anyone of ordinary skill in the art will readily appreciate that many variations and alterations to the following exemplary details are within the scope of the invention. Accordingly, the following preferred embodiment of the invention is set forth without any loss of generality to, and without imposing limitations upon, the claimed invention.

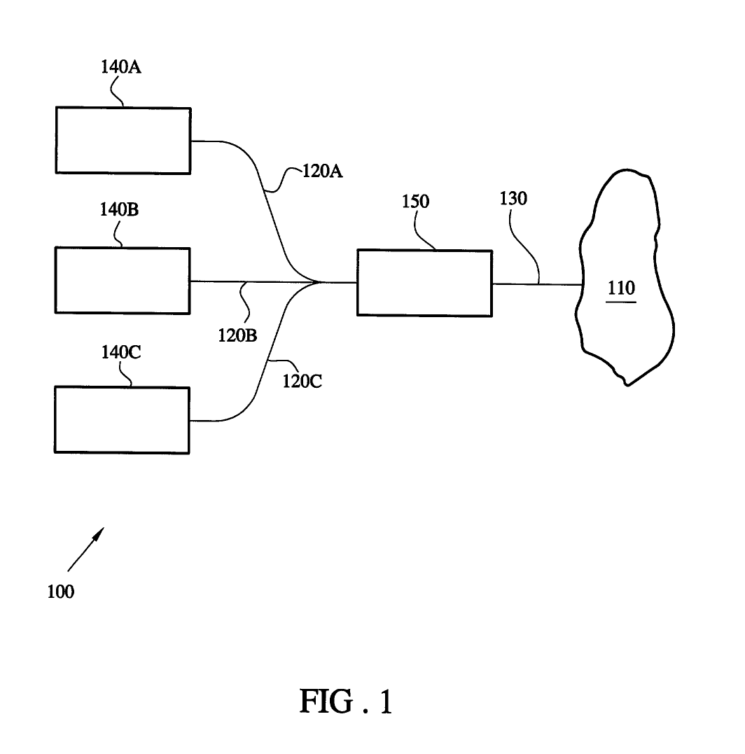

The present invention provides a multiple laser treatment apparatus and method 100, as shown by an exemplary embodiment in FIG. 1, that provides versatility and flexibility in treating a substance 110 with multiple laser treatment beams 120A, 120B and 120C at the same time. In general, the present invention provides an apparatus and method wherein two or more laser treatment beams, with each laser treatment beam having at least one distinct laser beam parameter, are selected and delivered simultaneously in a combined laser treatment beam 130 to substance 110 at which substance 110 undergoes treatment. Combined laser treatment beam 130 is also referred to as combined beam 130. The delivery of combined beam 130 enables one to apply two or more different treatments at the same time to substance 110 instead of just one single treatment each time as is most common in the prior art. Treatment in the present invention is then defined as a combination of two or more different laser treatment beams applied simultaneously. Treatment is also referred to as photodynamic therapy. The type of treatment is dependent on substance 110 and the structural change of substance 110 that one wants to achieve. Substance 110 could be any type of substance, but is preferably a substance with different compositions or structures such as, but not limited to, biological tissue, (bio)chemical compounds, bioengineering compositions and physical structures or materials. However, the present invention is not limited to these structures as it could include food products or fluids. In case of biological tissue, combined beam 130 is, for instance, applied in surgical or endoscopic surgery wherein different cells or tissue are treated with different laser treatment beams 120A, 120B to 120C and simultaneously delivered to substance 110 by combined beam 130. Examples of surgical or endoscopic surgery are, for instance, but not limited to, dermatological, urological (prostate), myringotomy, cardiovascular, neurological, otolaryngological, or visual procedures. In case of (bio)chemical compounds, combined beam 130 is, for instance, applied in genetic engineering wherein different laser treatment beams 120A, 120B to 120C could alter different parts of DNA as they are simultaneously delivered to substance 110 by combined beam 130. In case of materials, combined beam 130 is, for instance, applied in material engineering or semiconductor applications, wherein different laser treatment beams 120A, 120B to 120C simultaneously alter various parts of the structure as they are delivered to substance 110 by combined beam 130. As one skilled in the art might readily appreciate, various different examples could be developed and the present invention is not limited to the above mentioned examples.

The example shown in FIG. 1 includes three lasers 140A, 140B and 140C, however, the present invention generally includes two or more lasers. Each laser simultaneously delivers a laser treatment beam. Each laser treatment beam has at least one distinct laser beam parameter. The lasers can be can be different lasers or the same type of lasers. In case of the same type of laser at least one laser beam parameter in each laser treatment beam is different. In general, one or more laser beam parameters of the laser treatment beams are different. However, one or more laser beam parameters of the laser treatment beams can also be the identical. Several different types of lasers could be employed, such as, but not limited to, different type gas lasers (such as CO₂, excimer, argon, cu— vapor lasers), flashlight laser, liquid lasers (dye lasers) or solid state lasers (such as YAG, semiconductor, Ti:sapphire lasers). The present invention is not limited to a pulsed laser or a continuous wave laser. Coherent Inc. provides a product line with a wide variety of diode lasers that each have a different wavelength or wavelength range. For instance, Coherent's product line encompasses continuous wave (CW) laser diode bars, single stripe CW, conduction cooled quasi continuous wave (QCW) laser diode bars, fiber array packaged bars, or all kinds of integrated packages. In addition, Coherent's product line of Sapphire lasers (e.g. the solid state 488 nm laser) could be used.

Each laser can be controlled or programmed to select and deliver different laser treatment beams 120A, 120B to 120C simultaneously. In the present invention, each laser treatment beam has at least one distinct laser beam parameter. The different laser treatment beams are combined by delivery means 150 into combined beam 130. Combined beam 130 is delivered at substance 110. Each laser treatment beam 120A, 120B to 120C could be transmitted to and from delivery means 150 by any type of suitable optical path. Examples of optical paths that could be used are, for instance, but not limited to, optical fibers, articulated arms or waveguides. As is described in detail below, delivery means 150 could, for instance, include an optical device, a micromanipulator or a mirror-based optical delivery device. Combined beam 130 could either be directly delivered by delivery means 150 to substance 110 or could be further transmitted by, for instance, an optical fiber or a waveguide inside substance 110 as is, for instance, but not limited to, useful in endoscopic procedures.

Laser beam parameters are, for instance, but not limited to, wavelengths ranging from ultraviolet to far infrared, fluences, power levels, energy levels, temporal parameters, geometrical parameters, spot size, linear delivery parameters or three-dimensional delivery parameters. As one skilled in the art might readily appreciate, the present invention provides a platform to advance treatment plans or recipes with a combination of two or more laser treatment beams for simultaneous delivery to substance 110. An example of some laser beam parameters as known in the prior art for some exemplary complaints or treatments are shown in Table 1 which is illustrative rather than restrictive. Table 1 shows spot size, energy level and wavelength as exemplary laser beam parameters for these exemplary complaints or treatments as they are currently used in single laser beam treatments.

TABLE 1
Examples of some laser beam parameters for
some exemplary complaints or treatments.

| Lesion | Spot Size | Energy | | Wavelength |
|---|---|---|---|---|
| Telangiactasia's | 5 mm | 8-10 | J/cm² | 595 nm |
| Facial | 7 mm | 6-7.5 | J/cm² | 595 nm |
| | 2 × 7 mm | 11-16 | J/cm² | 595 nm |
| Rosacea or | 2 × 7 mm | 18-25 | J/cm² | 595 nm |
| Erythrosis Leg | 2 × 7 mm | 19-26 | J/cm² | 600 nm |
| | 5 mm | 10-12 | J/cm² | 595 nm |
| Matting | 5 mm | 12-14 | J/cm² | 600 nm |
| | 7 mm | 6-7 | J/cm² | 595 nm |
| | 10 mm | 4 | J/cm² | 595nm |
| Scars | 6 mm | 10-12 | J/cm² | 595 nm |
| | 7 mm | 5-8 | J/cm² | 585, 595 nm |
| | 10 mm | 4-6 | J/cm² | 595 nm |
| Striae | 7 mm | 5-7 | J/cm² | 595 nm |
| | 7 mm | 4-5 | J/cm² | 585, 595 nm |
| | 10 mm | 3-5 | J/cm² | 585, 596 nm |
| Adult PWS | 5 mm | 12-14 | J/cm² | 585 nm |
| Face | 5 mm | 14-16 | J/cm² | 585 nm |
| Trunk | 5 mm | 10-15 | J/cm² | 585, 595 nm |
| Port Wine Stain | 7 mm | 6-7 | J/cm² | 595 nm |
| (head/neck) | 7 mm | 8-9 | J/cm² | 595 nm |
| | 10 mm | 8 | J/cm² | 585, 595 nm |
| | 5 mm | 10-12 | J/cm² | 595, 600 nm |
| Hemangiomas | 7 mm | 6-8 | J/cm² | 595, 600 nm |
| | 10 mm | 8 | J/cm² | 585 nm |
| Angioma or | 5 mm | 8-10 | J/cm² | 595 nm |
| Spider Angioma | 7 mm | 6-8 | J/cm² | 595 nm |
| | 10 mm | 4-5 | J/cm² | 595 nm |

As one skilled in the art might readily appreciate, a large number of combinations of laser beam parameters could be derived even if just two of the same lasers are used. An example is, for instance, that two of the same lasers are used each delivering a laser treatment beam with the same wavelength, however, each laser treatment beam is delivered at a different power level; e.g. laser 1 could use only 10% of the power and laser 2 could only use 90% of the power. Another example, is that two of the same lasers are used each delivering a laser treatment beam with the same wavelength, however, each laser treatment beam is delivered with different geometrical beam parameters. Geometrical beam parameters are, for instance, but not limited to, the diameter of a beam, the focus point of a beam or the de-foci footprint(s) of a beam.

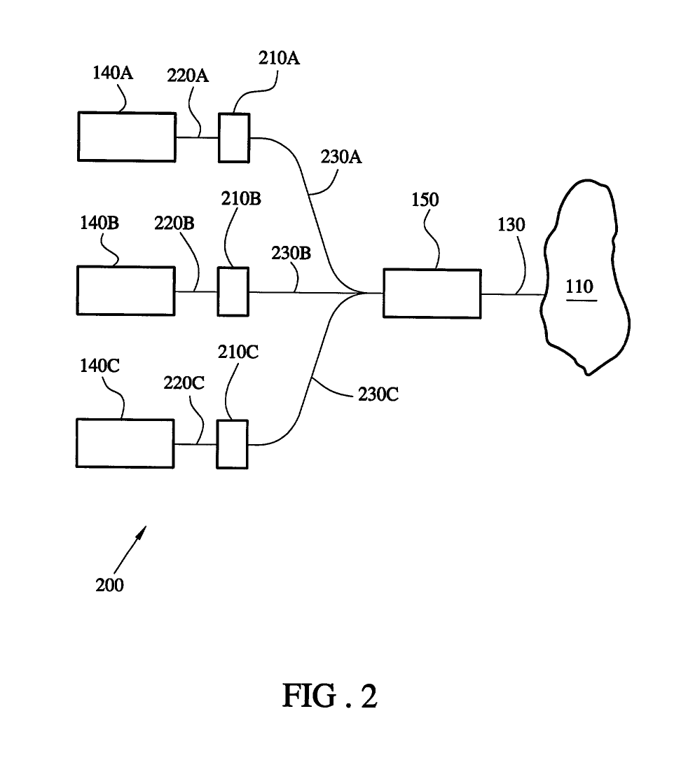

There are several different ways to select, by adjusting or controlling, the laser beam parameters. For example, laser beam parameters can be selected by adding hardware components, such as one or more optical elements, to apparatus and method 100 to change a laser beam parameter. FIG. 2 shows an exemplary embodiment of a multiple laser treatment apparatus 200 that is similar to in FIG. 1 with the addition of optical components 210A, 210B and 210C that could select by adjusting or controlling one or more beam parameters of laser treatment beams 220A, 220B and 220C into selected laser treatment beams 230A, 230B and 230C, respectively. FIG. 2 shows one optical component for each laser treatment beam, but there is no limitation to the number of optical components that could be used to select a laser beam parameter. Examples of optical components 210A, 210B and 210C include, for instance, but are not limited to, a collimator, a spherical element, an a-spherical element, a parabolic element, or any other optical element that could adjust the beam parameter of the laser treatment beam.

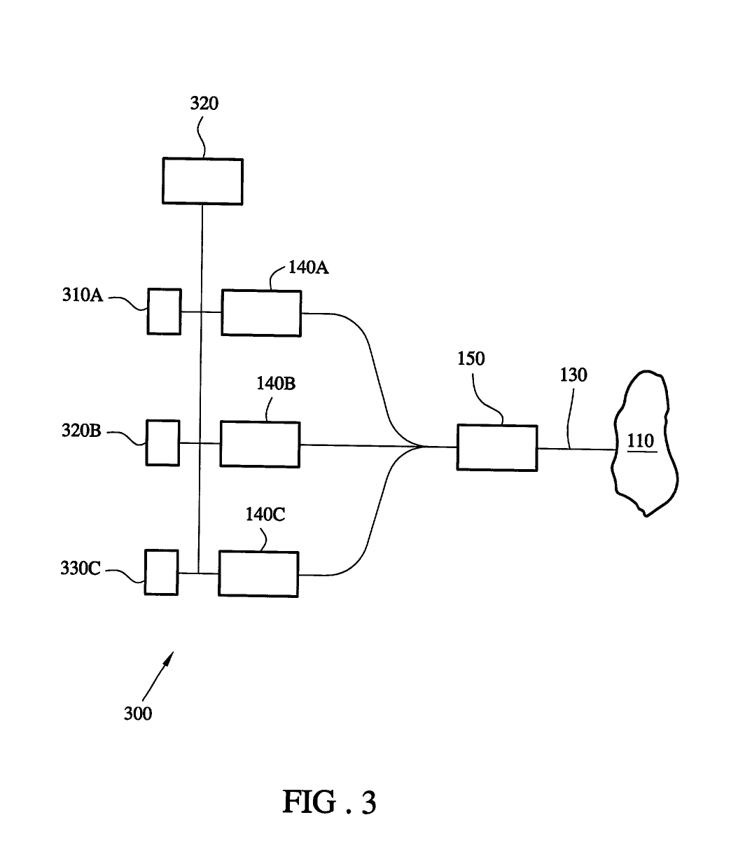

Another way to select laser beam parameters is by a control means that allows one to control, for instance, one or more lasers or one or more laser beam parameters. FIG. 3 shows an exemplary embodiment of a multiple laser treatment apparatus and method 300 that is similar to FIG. 1 with the addition of control means 310A, 310B and 310C that are linked directly to laser 140A, 140B and 140C respectively, or control means 320 that is a single control panel that is linked to all lasers 140A, 140B and 140C. Examples of control means include, for instance, but are not limited to, a software panel or interface with virtual control panels and buttons or a hardware panel with control buttons. Control means 310A, 310B and 310C or control means 320 enables a user to control, for instance, but not limited to, the selection of wavelengths, the energy (or fluence) of each wavelength, the intensity (or power), the temporal parameters (such as pulse parameters and repetition rate) of each laser treatment beam and the repetition rate of the combination of wavelengths or each individual wavelength.

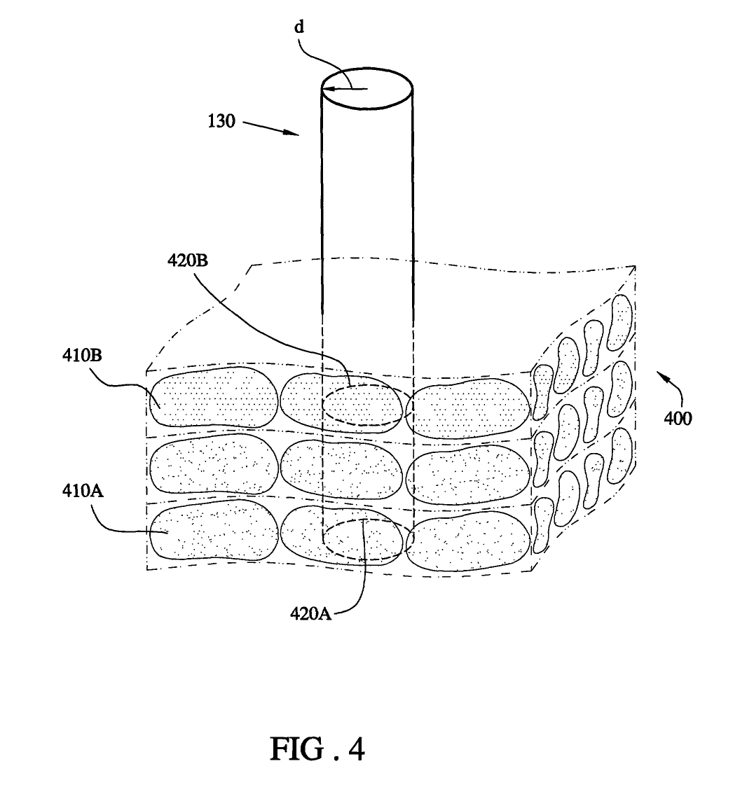

As mentioned above, a large number of different laser treatment beams could be combined. FIGS. 4-7 show some illustrative examples of two different laser treatment beams in combined beam 130. FIG. 4 shows an example of substance 400 that could, for instance, be a biological tissue with different cells 410A and 410B. The treatment plan may require a combined beam 130 that includes two laser treatment beams 420A and 420B. The wavelength and tissue penetration depth are different for laser treatment beam 420A and 420B. For instance, laser treatment beam 420A targeting cells 410A is delivered by a Ruby laser with a wavelength of 694 nm and laser treatment beam 420B targeting cells 410B is delivered by a Er:YAG laser with a wavelength of 2940 nm. Laser treatment beams 420A and 420B in combined treatment beam 130 have similar geometrical parameters as shown by diameter d of laser treatment beams 420A and 420B.

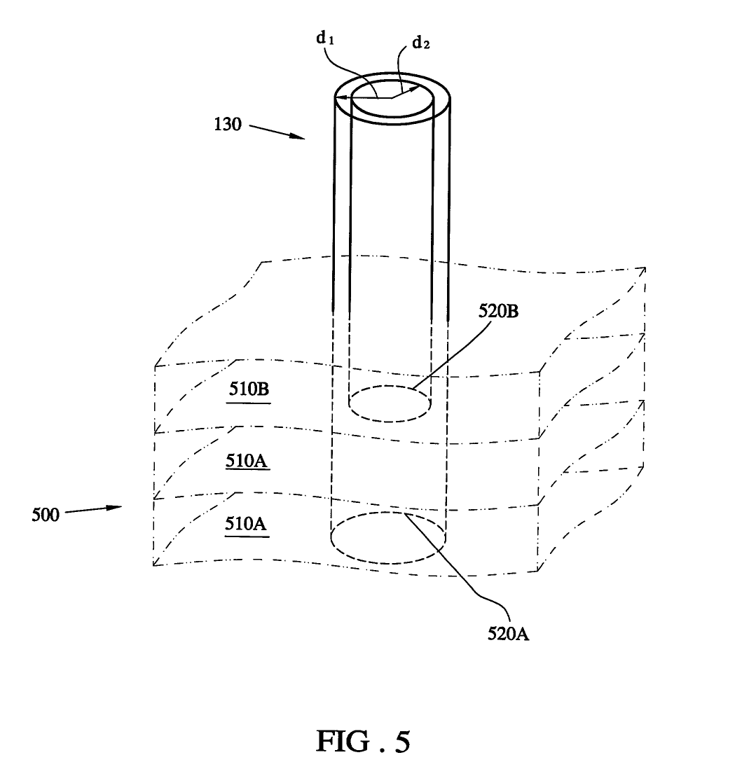

FIG. 5 shows an example of substance 500 that could, for instance, be a biological tissue with different tissue layers 510A and 510B. The treatment plan may require a combined beam 130 that includes two laser treatment beams 520A and 520B. In this example, the wavelength, tissue penetration depth as well as laser beam diameter are different for laser treatment beams 520A and 520B. For instance, laser treatment beam 520A targeting tissue 510A is delivered by a CO₂ laser with a wavelength of 10,600 nm and laser treatment beam 520B targeting tissue 510B is delivered by a Alexandrite laser with a wavelength of 755 nm. Laser treatment beam 520A and 520B in combined beam 130 have different geometrical parameters as shown by diameter d₁ of beam 520A and diameter d₂ of beam 520B.

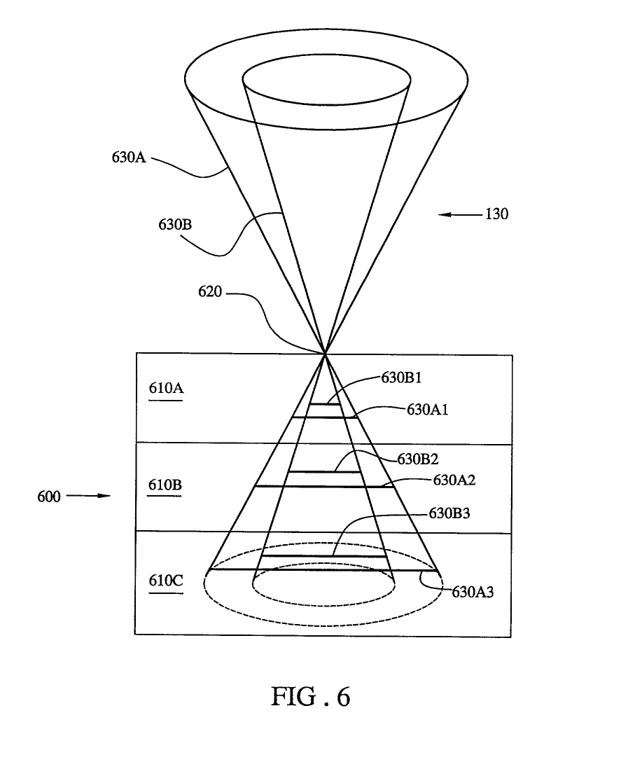

FIG. 6 shows an example of substance 600 that could, for instance, be a physical structure with different materials 610A, 610B and 610C. The treatment plan may require a similar focus point 620 of laser treatment beams 630A and 630B that are combined in combined beam 130. However, the key aspect of this particular treatment might be to have different de-foci footprints 630A1, 630A2 and 630A3 for laser treatment beam 630A and 630B1, 630B2 and 630B3 for laser treatment beam 630B.

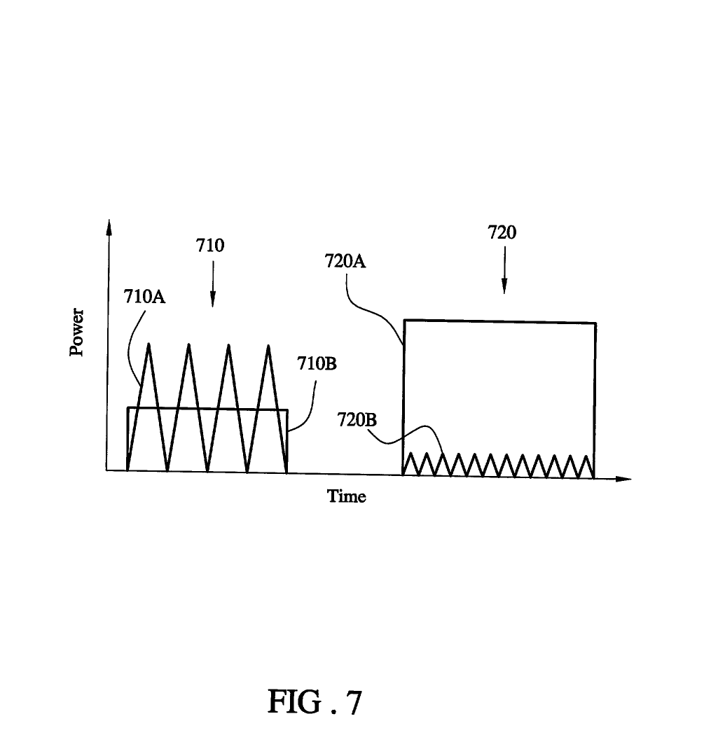

FIG. 7 shows an example of two combined laser treatment beams 710 and 720 wherein the laser treatment beams have different temporal parameters. Temporal parameters of a laser treatment beam are, for instance, but not limited to, the pulse repetition rate, duration of the pulse and overall radiation time of the laser treatment beam. For example, combined treatment beam 710 has a high repetition, high power beam 710A and a low power, continuous beam 710B. In the other example, combined treatment beam 720 has a long pulse, high power beam 720A and a short pulse, low power beam 720B.

As mentioned above, delivery means 150 could, for instance, include a micromanipulator (e.g. micromanipulator 710/711 Acuspot by Sharplan Lasers Inc., micromanipulator by TTI Medical Inc. or Cryomedics micromanipulator by Cabot Medical Inc.), an optical device or a mirror-based optical delivery device. The preferred delivery means 150 is a device that preserves the mode of each of the laser treatment beams.

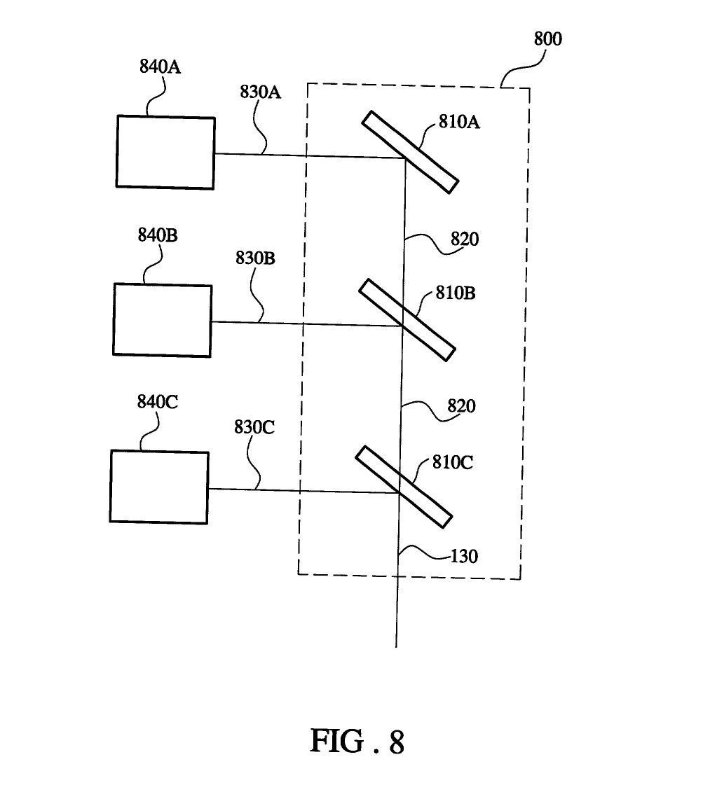

FIG. 8 shows an exemplary embodiment of delivery means 150 that includes an optical device 800. FIG. 8 shows optical components 810A, 810B and 810C that are aligned on an optical path 820 to receive laser treatment beams 830A, 830B and 830C from lasers 840A, 840B and 840C respectively. Each optical component 810A, 810B and 810C directs and selectively combines laser treatment beams 830A, 830B and 830C along optical path 820. Examples of the various kinds of optical components that can be used are, for instance, a wavelength selective mirror, a wavelength selective filter, a beam splitter, or any other optical device that is capable of directing and selectively combining different laser treatment beams that are selected to create combination 130. An illustrative example of such a mirror is, without being restrictive, a Silflex MK-II mirror by Unaxis-Balzers Inc. This mirror has high reflectivity values through the visual, near, middle and far infra red. Optical device 800 could further include position or rotation means (not shown) to control the linear position or angular position of optical components 810A, 810B and 810C with respect to optical path 820. Position or rotation means could be established by various different techniques such as, for instance, an optical switching device, a folding beam splitter, a piezo-electric element, a solenoid, a preprogrammed stepper motor, or the like. Positioning of optical components 810A, 810B and 810C is, for instance, related to removing an optical component away from the optical path if the optical component was already positioned in the optical path. A reason for removing an optical component is, for instance, based on a selection by a user that the particular laser treatment beam outputted by the corresponding laser is no longer necessary in the selected combination or possibly interferes with the selected combination. Rotating optical components 810A, 810B and 810C is, for instance, related to re-direct one or more laser treatment beams to generate a subset of combinations of the laser treatment beams. Position or rotation means is also meant for aligning or re-aligning optical components 810A, 810B and 810C along optical path 820.

Since the present invention involves a combination of laser treatment beams each having different laser beam parameters, a lens based system to deliver combined beam 130 would not only be impractical, but would also cause chromatic aberration. In addition, a lens based system cannot be focused to a spot size smaller than 0.4 mm. Therefore in order to more practically and more accurately focus combined beam 130 on a desired spot, it is necessary that delivery means 150 includes a mirror-based optical delivery device to control the focus of combined beam 130 on substance 110. U.S. Pat. Nos. 5,955,265 and 5,163,936 (both hereby incorporated by reference) assigned to the same assignee as the present invention, discloses a mirror-based optical delivery device that was invented to avoid chromatic aberration and better focus a laser beam by aligning a visual beam with the laser beam wherein the visual beam is solely used to visually guide the laser beam. The mirror-based optical delivery device is preferred as delivery means 150 in the present invention to more practically and more accurately focus combined beam 130 at substance 110. In the present, invention mirror-based optical delivery device delivers and controls two or more different laser treatment beams to substance 110.

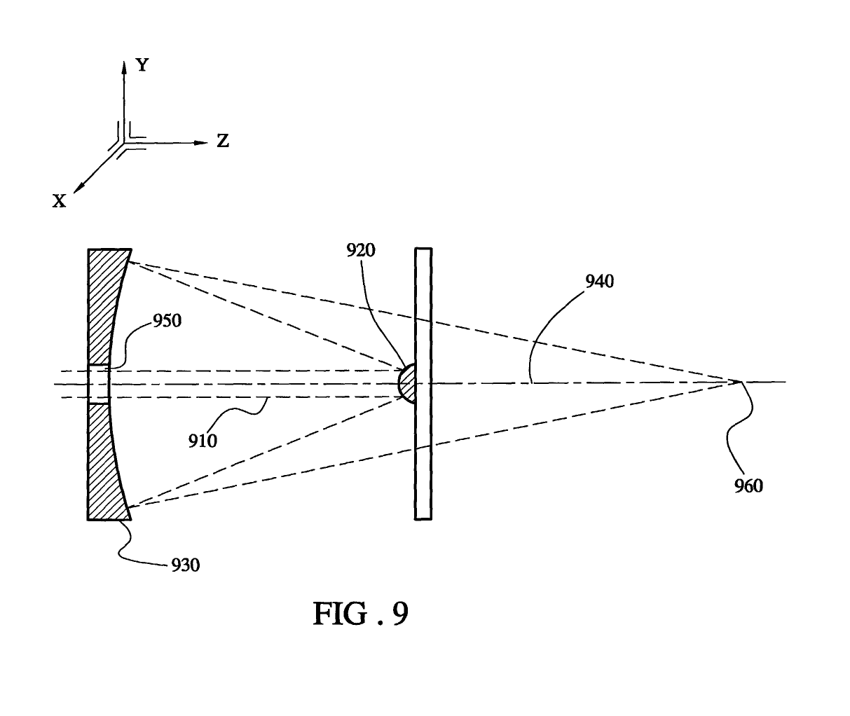

U.S. Pat. No. 5,128,509 (hereby incorporated by reference) to the present inventor and assigned to the same assignee as the present invention discloses a mirror-based optical delivery device 900 as shown in FIG. 9, which uses reflective optics to steer and focus combined laser beam 910. The optical focusing of device 900 is performed by a convex mirror 920 and a concave mirror 930 facing each other and aligned on a common optical axis 940. Combined laser beam 910 passes through a small hole 950 in the center of concave mirror 930 and is reflected by convex mirror 920 back towards concave mirror 930. Concave mirror 930 reflects the beam forward to a focus 960 beyond convex mirror 920. Because this device uses reflective optics, it is capable of delivering laser treatment beams of a wide range of wavelengths and laser beam parameters and to a very small focus. With the mirror-based optical delivery device, the present invention enables one to deliver combined beam on substance with a spot size that is 0.1 mm or less. Alternatively, the present invention is not restricted to allow one to deliver combined treatment beam on substance with a spot size that is 0.1 mm or more. Unlike systems using refractive optics, mirror-based optical delivery device 900 enables one to simultaneously deliver coincident laser treatment beams ranging from ultraviolet to far infrared. Moreover, because reflective optics do not exhibit chromatic aberration, mirror-based optical delivery device 900 delivers combined beam 910 with two or more laser treatment beams to the same focal point.

Mirror-based optical delivery device 900, however, does not provide a means for scanning to produce a uniform exposure over a large surface area. U.S. Pat. No. 5,995,265 to the present inventor and assigned to the same assignee as the present invention discloses a mirror-based optical delivery device with linear scanning or delivery means to scan a treatment area with a predetermined linear scanning or delivery pattern. To establish a linear scanning or delivery pattern different control means (not shown) are included to rotate concave mirror 930 and/or convex mirror 920 around the X, Y and/or Z-axis. In the present invention, laser treatment beams delivered in combined treatment beam could generate various different kinds of treatment patterns, such as a spiral treatment pattern to cover an elliptical region rather than a circular one. In addition, the treatment pattern can be adjusted to cover annular regions and elliptically annular regions. The treatment pattern can also be adjusted so that the combined beam follows a circular or elliptical path rather than a spiral path. The path can also be adjusted to follow other types of paths, such as a Lissajous figure. Of course, by fixing the mirrors, combined beam may be directed to a single point as well. Since the path of combined beam could be controlled by a microprocessor programming device or by hand, the types of paths and treatment patterns are not limited to any single class.

U.S. Pat. No. 5,995,265, however, does not teach a means for three-dimensional scanning to produce a depth exposure over a large area. The present invention further includes a mirror-based optical delivery device with three-dimensional scanning or delivery means to treat a three-dimensional area with a three-dimensional scanning or delivery pattern. An example of how a three-dimensional scanning or delivery pattern could be established is, for instance, by combining linear scanning means as described above with a control means (such as one or more stepper motors, not shown) that is capable of changing the relative position of convex mirror 920 and a concave mirror 930 along common optical axis 940, i.e. to translate concave mirror 930 and convex mirror 920 over the Z-axis with respect to each other. As one skilled in the art might readily appreciate, several different ways could be developed to control the relative position between convex mirror 920 and a concave mirror 930 along common optical axis 940. The path of combined beam 910 could be controlled by a microprocessor programming device or by hand which enables one to create any type of three-dimensional paths and treatment pattern. The delivery means of the present invention allows one to deliver a treatment pattern in a static manner or in a dynamic manner where the three-dimensional treatment patterns changes shape and location at the substance during the treatment.

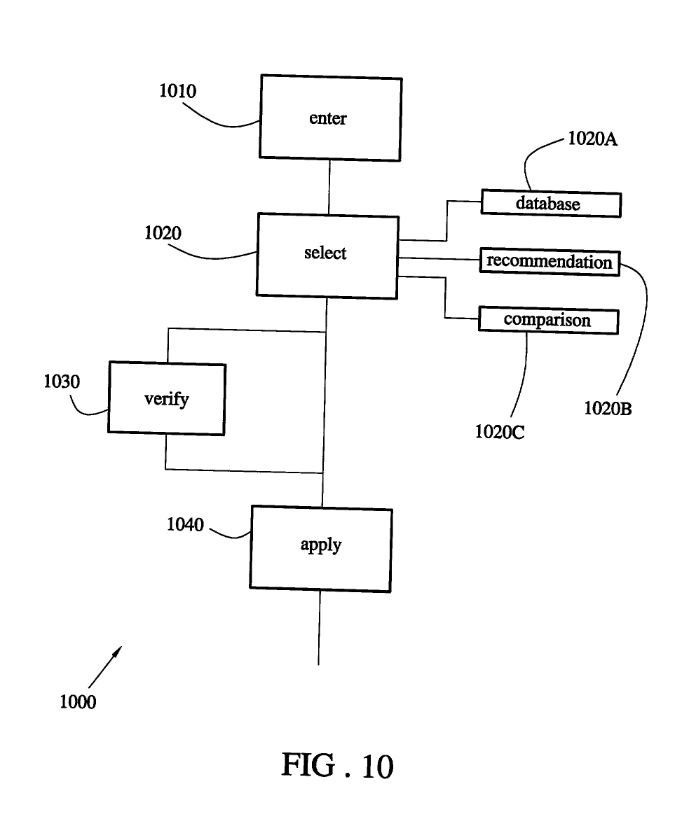

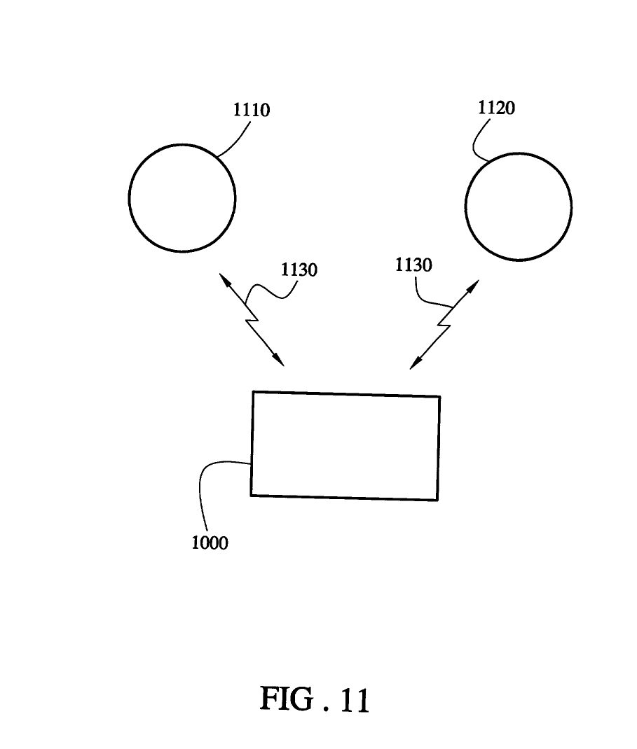

FIG. 10 shows a computer program 1000 to manage and control the simultaneously delivering multiple laser treatment beams to a substance with a laser treatment system. Computer program 1000 can be implemented by a variety of computer programs or means such as C⁺⁺, Java, Unix, HTML, XML and the like. Computer program 1000 can also be implemented on different hardware devices, such as computer devices, handheld devices and the like. Computer program 1000 provides means to enter data 1010. Means to enter data are, for instance, but not limited to, a keyboard, a touch-screen, a handheld device, a web-based application, a voice recognition system and the like. Computer program 1000 is not limited to any other means for entering data. The type of data that can be entered is, for instance, but not limited to, the type of lasers, the type of laser treatment beams, laser beam parameters, substance information, treatment protocols, complaint information, disease information, etc. In the example of a patient that needs to undergo a laser treatment, data can also include patient information data including patient visits and type of previous or related treatments. Computer program 1000 also provides means for selecting a treatment plan 1020. Means for selecting 1020 are, for instance, but not limited to, through a keyboard, a touch-screen, a handheld device, a web-based interaction, a voice recognition system and the like. Computer program 1000 can select a treatment plan from a database 1020A that contains, for instance, predetermined treatment plans. A treatment plan can also be selected based on a recommendation 1020B of a treatment plan which is based on, for instance, previous treatment trials or intelligent reasoning, or comparison 1020C based on entered data 1010. Guidance or recommendation is established by having knowledge stored in a database that can be accessed or requested from the computer program. The computer program could then respond by providing a list of choices and recommendations after which the user could either select or modify the provided choices and subsequently perform the procedure. Once the treatment plan has been established, the user has the opportunity to verify 1030 the selected treatment plan before it is applied 1040 to the substance. Different means for verifying 1030 the treatment plan could be used such as, for instance, but not limited to, visual inspection of the list of laser beam parameters, including boundaries and/or warnings for the laser beams parameters or combination of laser treatment beams, statistical verifications or calculations and the like. The verifying means 1030 is not limited to verifying the combined treatment beam before it is applied to the substance since it can also be verified in simulation or virtual environment. The user could also verify the combined beam by actually applying combined beam at a test substance. The user could also elect to have verifying means as an optional step in computer program 1000. This optional step makes most sense if the treatment is a standard approach and used on a routine basis. Means to apply 1040 the combined treatment beam encompasses any software or hardware connection that allows the program to control the multiple laser treatment apparatus. These type of connections are well known in the art. Computer program 1000 includes different ways of communicating 1130 data or information as shown in FIG. 11 between a user or an another computer, indicated by remote station 1110 and 1120. Remote station 1110 and 1120 could, for instance, contain a useful database, new information for treatment plans, mailing list information, software updates or any other useful information for the laser treatment plan. Means of communicating are, for instance, but not limited to, wireless communication means or any type of conventional communication means to communicate data as they are known in the art. Useful information, related to laser treatment plans wherein two or more laser treatment beams are delivered simultaneously, could be stored in a database. The database could, for instance, be accessed by computer program 900. Such a database provides information of a plurality of treatment plans that specifies the type of lasers and laser beams parameters. As one skilled in the art would readily appreciate, such a database could include various kinds of related parameters such as substance-related information, patient-related information, etc. In general the type of data in the database depends on the type of treatment plan which varies from any type of medical treatment plan, any type of photodynamic therapy, any type of (bio)chemical or bioengineering treatment plan or any type of physical treatment plan. Furthermore, the database could also contain a variety of treatment or diagnostic maps as is described below.

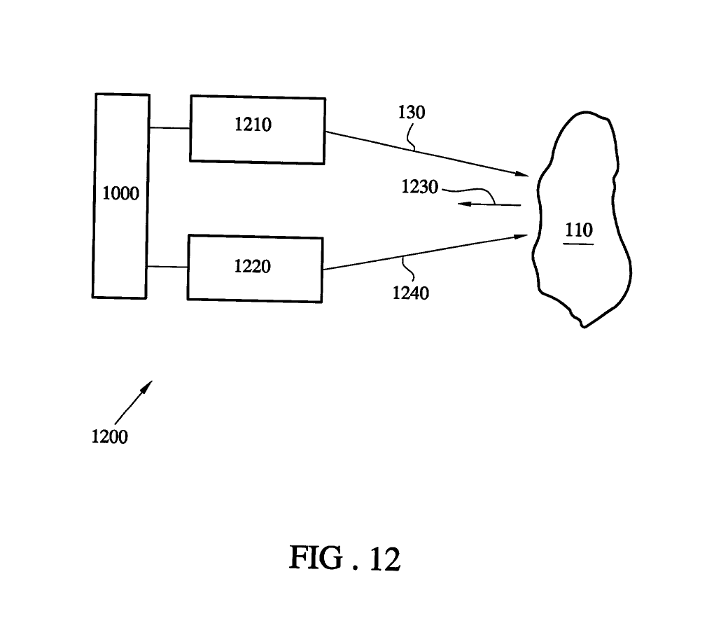

Another example to recommend a treatment plan to computer program 1000 as shown in FIG. 10 is by including a diagnosing means 1220 that uses fluorescence emission 1230 to the multiple laser treatment apparatus and method 1210 of the present invention as shown by 1200 in FIG. 12. Diagnosing means 1210 could either be a separate module or an integral part of the multiple laser treatment apparatus and method 1210 of the present invention. Diagnosing means 1220 includes a diagnostic system that enables a user to map an area of the substance using fluorescent emission 1230 as a result of delivering a laser diagnostic beam 1240 to the substance. Such a map could be stored in the database and accessed by computer program 1000. Computer program 1000 then further includes means to interpret and/or analyze the fluorescence maps in terms of a pattern or geographical map. Once the pattern or map has been analyzed, the computer program could be further employed to diagnose a particular complaint, disease or deformation of substance. Computer program 1000 could access a database of patterns to allow for a comparison and/or analysis of the detected pattern with one or more patterns in the database. Such a comparison or analysis could either be done automatically by, for instance, pixel comparison or manually where a graphical user interface enables the user to perform such an comparison or analysis. Diagnosis means further includes means to allow the computer program to also recommend and/or (automatically) execute a treatment protocol by selecting the appropriate combination of laser treatment beams. The present invention could include any type of diagnostic means to provide data or recommendation. A preferred diagnostic means 1220 is a multiple laser diagnostic apparatus and method wherein two or more laser diagnostic beams are used to diagnose a substance. Details regarding such a multiple laser diagnostic apparatus and method is disclosed in copending U.S. patent application entitled “Multiple Laser Diagnostics” by the same inventor as the present invention and having the same filing date as the present invention. This copending application is incorporated by reference for all that is discloses.

The present invention has now been described in accordance with several exemplary embodiments, which are intended to be illustrative in all aspects, rather than restrictive. Thus, the present invention is capable of many variations in detailed implementation, which may be derived from the description contained herein by a person of ordinary skill in the art. For instance, the apparatus of the present invention could easily be developed as a handheld delivery apparatus. This handheld delivery apparatus is preferably portable and transferable to enable one to use the apparatus at various different places and circumstances. A preferred embodiment of handheld delivery apparatus is a miniature handheld delivery apparatus with dimensions of 6″ by 12″ by 20″ or less. Furthermore, the handheld delivery apparatus could be fully operational by independent power such as battery power. In addition, many different optical components can be used to select or establish the desired combination of laser treatment beams. The present invention could include different means as part of the delivery means to preserve the mode of each laser treatment beam. In addition, the present invention also includes means to vary or continuously change the pattern of the laser beams during the performance of a treatment. The present invention could be used in many different applications including other (bio)medical, bioengineering and industrial applications. A variety of computer programs, environments and user interfaces can be used to control the various hardware and software components that encompasses the present invention. In addition, various kinds of display mechanism can be used and are not restricted to head-sets and glasses (see e.g. U.S. Pat. Nos. 5,114218, 5,151,600, 5,184,156 and 5,382,986 all assigned to the same assignee as the present invention) or flat panel devices to give the user control and feedback of the treatment protocol and procedure. All such variations are considered to be within the scope and spirit of the present invention as defined by the following claims and their legal equivalents.

What is claimed is:

1. A multiple laser treatment apparatus, comprising:
(a) n lasers, wherein said n>1 and each of said n lasers simultaneously delivers a laser treatment beam selected for a treatment wherein each one of said laser treatment beams comprises at least one different laser beam parameter; and
(b) means to deliver said laser treatment beams in a combined treatment beam wherein said combined treatment beam is delivered at a substance at which said substance undergoes said treatment.

2. The apparatus as set forth in claim 1, wherein said laser beam parameters are wavelengths, fluences, power levels, energy levels, temporal parameters, geometrical parameters, spot sizes, linear delivery parameters or three-dimensional delivery parameters.

3. The apparatus as set forth in claim 2, wherein said wavelength is selected from a spectrum of wavelengths ranging from ultraviolet to far infrared.

4. The apparatus as set forth in claim 2, wherein said one or more laser beam parameters of said laser treatment beams are different.

5. The apparatus as set forth in claim 2, wherein said one or more laser beam parameters of said laser treatment beams are identical.

6. The apparatus as set forth in claim 1, further comprising at least one optical component to select one or more laser beam parameters of one or more of said laser treatment beams.

7. The apparatus as set forth in claim 6, wherein said optical component is a beam profiler, a collimator, a spherical element, an a-spherical element or a parabolic element.

8. The apparatus as set forth in claim 1, further comprising means to control each one of said n lasers.

9. The apparatus as set forth in claim 8, wherein said means to control comprises a single control panel.

10. The apparatus as set forth in claim 1, further comprising means to control one or more laser beam parameters of at least one of said laser treatment beams.

11. The apparatus as set forth in claim 1, wherein one or more of said n lasers is a gas laser, liquid laser, solid state laser, semiconductor diode laser, a tunable laser or a flashlight laser.

12. The apparatus as set forth in claim 1, further comprising at least one optical path to transmit said laser treatment beams, wherein said optical path is an optical fiber, an articulated arm or a waveguide.

13. The apparatus as set forth in claim 1, wherein said means to deliver comprises a mirror-based optical delivery system to control said combined treatment beam.

14. The apparatus as set forth in claim 13, wherein said mirror-based optical delivery system has a spot size of 0.1 mm or less.

15. The apparatus as set forth in claim 13, wherein said mirror-based optical delivery device has a spot size of 0.1 mm or more.

16. The apparatus as set forth in claim 13, wherein said optical delivery device comprises linear delivery means.

17. The apparatus as set forth in claim 13, wherein said optical delivery device comprises three-dimensional delivery means.

18. The apparatus as set forth in claim 1, wherein said means to deliver comprises a micromanipulator.

19. The apparatus as set forth in claim 1, wherein said means to deliver comprises endoscopic delivery means.

20. The apparatus as set forth in claim 1, wherein said means to deliver comprises an optical device wherein said optical device comprises:
(a) n optical components aligned on an optical path to receive said laser treatment beams from said n lasers, wherein said laser nᵢ corresponds to said optical component nᵢ and i=1, . . . n, and wherein each of said n optical components directs and selectively combines said laser treatment beams of said n lasers along said optical path; and
(b) an optical delivery system connected to said optical path to deliver said combined treatment beam to said substance.

21. The apparatus as set forth in claim 20, wherein one or more of said n optical components is a wavelength selective mirror, a beam splitter or a wavelength selective filter.

22. The apparatus as set forth in claim 20, further comprising means to position said n optical components in said optical path or away from said optical path.

23. The apparatus as set forth in claim 20, further comprising position means to generate a subset of combinations of said laser treatment beams.

24. The apparatus as set forth in claim 1, wherein said substance is a biological tissue, a chemical compound, a biochemical compound, a food product, a fluid, a bioengineering composition or a physical structure.

25. The apparatus as set forth in claim 1, wherein said treatment is a medical treatment and said laser treatment beams are medically useful treatment beams.

26. The apparatus as set forth in claim 1, further comprising means for diagnosing said substance.

27. The apparatus as set forth in claim 26, wherein said diagnosing means comprises a diagnostic system, wherein said diagnostic system maps an area of said substance using fluorescent emission.

28. The apparatus as set forth in claim 1, wherein said apparatus is a handheld delivery apparatus.

29. The apparatus as set forth in claim 28, wherein said handheld delivery apparatus is a portable and transferable miniature handheld delivery apparatus with dimensions of 6″ by 12″ by 20″ or less.

30. The apparatus as set forth in claim 1, wherein said apparatus operates on independent power.

31. A multiple laser treatment apparatus, comprising:
(a) means to select two or more laser treatment beams selected for a treatment wherein each one of said laser treatment beams comprises at least one different laser beam parameter; and
(b) means to simultaneously deliver said laser treatment beams in a combined laser treatment beam at a substance at which said substance undergoes said treatment.

32. The apparatus as set forth in claim 31, wherein said laser beam parameters are wavelengths, fluences, power levels, energy levels, temporal parameters, geometrical parameters, spot sizes, linear delivery parameters or three-dimensional delivery parameters.

33. The apparatus as set forth in claim 31, wherein said means to select comprises at least one optical component to select one or more of said laser beam parameters of one or more of said laser treatment beams.

34. The apparatus as set forth in claim 31, wherein said means to select comprises means to control said laser beam parameters.

35. The apparatus as set forth in claim 31, wherein said means to deliver comprises a mirror-based optical delivery system to control said combined treatment beam.

36. The apparatus as set forth in claim 35, wherein said mirror-based optical delivery system has a spot size of 0.1 mm or less.

37. The apparatus as set forth in claim 35, wherein said mirror-based optical delivery device has a spot size of 0.1 mm or more.

38. The apparatus as set forth in claim 35, wherein said optical delivery device comprises linear delivery means.

39. The apparatus as set forth in claim 35, wherein said optical delivery device comprises three-dimensional delivery means.

40. The apparatus as set forth in claim 31, wherein said means to deliver comprises a micromanipulator.

41. The apparatus as set forth in claim 31, wherein said means to deliver comprises endoscopic delivery means.

42. The apparatus as set forth in claim 31, wherein said means to deliver comprises an optical device to combine said laser treatment beams.

43. The apparatus as set forth in claim 31, further comprising means for diagnosing said substance.

44. The apparatus as set forth in claim 43, wherein said diagnosing means comprises a diagnostic system, wherein said diagnostic system maps an area of said substance using fluorescent emission.

45. A method for simultaneously delivering a combined laser treatment beam, comprising the steps of:
(a) selecting two or more laser treatment beams selected for a treatment wherein each one of said laser treatment beams comprises at least one different laser beam parameter; and
(b) simultaneously delivering said laser treatment beams in a combined laser treatment beam at a substance at which said substance undergoes said treatment.

46. The method as set forth in claim 45, wherein said laser beam parameters are wavelengths, fluences, power levels, energy levels, temporal parameters, geometrical parameters, spot sizes, linear delivery parameters or three-dimensional delivery parameters.

47. The method as set forth in claim 45, wherein said step of selecting comprises the step of providing at least one optical component to select one or more of said laser beam parameters of one or more of said laser treatment beams.

48. The method as set forth in claim 45, wherein said step of selecting comprises the step of controlling said laser beam parameters.

49. The method as set forth in claim 45, wherein said step of simultaneously delivering comprises the step of providing a mirror-based optical delivery system to control said combined treatment beam.

50. The method as set forth in claim 49, wherein said optical delivery device comprises linear delivery means.

51. The method as set forth in claim 49, wherein said optical delivery device comprises three-dimensional delivery means.

52. The method as set forth in claim 45, wherein said step of simultaneously delivering comprises the step of providing a micromanipulator.

53. The method as set forth in claim 45, wherein said step of simultaneously delivering comprises the step of providing endoscopic delivery means.

54. The method as set forth in claim 45, wherein said step of simultaneously delivering comprises the step of providing an optical device to combine said laser treatment beams.

55. The method as set forth in claim 45, further comprising the step of providing means for diagnosing said substance.

56. The method as set forth in claim 55, wherein said diagnosing means comprises a diagnostic system, wherein said diagnostic system maps an area of said substance using fluorescent emission.

57. A computer program to manage and control a simultaneous delivery of multiple laser treatment beams to a substance, comprising:
(a) means for selecting a treatment plan wherein said treatment plan comprises two or more laser treatment beams wherein each one of said laser treatment beams comprises at least one different laser beam parameter; and
(b) means for applying said treatment plan to said substance.

58. The computer program as set forth in claim 57, wherein said means for selecting comprises means for recommending said treatment plan.

59. The computer program as set forth in claim 57, wherein said means for selecting comprises a database of treatment plans.

60. The computer program as set forth in claim 57, wherein said means for selecting comprises means for comparing said treatment plan with a previous treatment plan.

61. The computer program as set forth in claim 57, further comprising means for entering data.

62. The computer program as set forth in claim 61, wherein said data comprises patient data, treatment plan data, or complaint or disease data.

63. The computer program as set forth in claim 57, further comprising means for verifying said treatment plan.

64. The computer program as set forth in claim 57, further comprising communication means to communicate information between said computer program and one or more remote stations.

65. A database of a plurality of laser treatment plans wherein two or more laser treatment beams are delivered simultaneously to a substance, comprising:
(a) said plurality of treatment plans; and
(b) said one or more laser beam parameters for each one of said treatment plans wherein each one of said laser treatment beams comprises at least one different laser beam parameter.

66. The database as set forth in claim 65, wherein said treatment plans are medical treatment plans, chemical treatment plans, biochemical treatment plans, bioengineering treatment plans or physical treatment plans.

67. The database as set forth in claim 65, further comprising substance-related information.

68. The database as set forth in claim 65, further comprising patient-related information.
